(19) United States
(12) Patent Application Publication
(10) Pub. No.: US 2014/0271894 A1
(43) Pub. Date: Sep. 18, 2014

(54) MATRIX-INCORPORATED FLUORESCENT POROUS SILICA PARTICLES FOR DRUG DELIVERY

(71) Applicant: INTERNATIONAL BUSINESS MACHINES CORPORATION, Armonk, NY (US)

(72) Inventors: Dylan J. Boday, Tucson, AZ (US); Jason T. Wertz, Wappingers Falls, NY (US)

(73) Assignee: INTERNATIONAL BUSINESS MACHINES CORPORATION, Armonk, NY (US)

(21) Appl. No.: 13/803,604

(22) Filed: Mar. 14, 2013

(51) Int. Cl. A61K 9/14

(52) U.S. Cl. CPC A61K 9/146
USPC 424/497; 424/490; 427/2.14

(57) ABSTRACT

A fluorescent porous silica particle for drug delivery includes a bridged silane fluorescent dye incorporated throughout the particle's matrix. Copolymerization of a bridged silane fluorescent dye (e.g., (R′O)₃Si—R—Si(OR′)₃, where R is a fluorescent organic bridging group, and where R′ is a methyl or ethyl group) and a tetralkoxysilane (e.g., Si(OR′)₄, where R′ is a methyl or ethyl group) in the presence of a surfactant generates matrix-incorporated fluorescent porous silica particles of a predetermined size and shape. A capping layer is then bonded onto the surface of each particle and, subsequently, the surfactant within the pores of each particle is removed. The capping layer reversibly changes between closed and opened states responsive to a stimulus. A payload is then loaded within of the pores by applying the stimulus to open the capping layer. The payload is then entrapped within the pores by removing the stimulus to close the capping layer.

### BACKGROUND OF THE INVENTION

1. Field of Invention

The present invention relates in general to the field of drug delivery. More particularly, the present invention relates to matrix-incorporated fluorescent porous silica particles for drug delivery, and preparation methods thereof.

2. Background Art

Fluorescent nanoparticles are becoming increasingly attractive in the medical field as drug delivery vehicles for use in vivo and in vitro. Typically, fluorescent nanoparticles are prepared using one of two approaches: core-shell encapsulation or particle surface modification. Unfortunately, these approaches each suffer one or more of the following limitations: unintended fluorescent dye leach out; inadequate dispersion of the fluorescent nanoparticle in aqueous media; and inability of the fluorescent nanoparticle to accommodate other functionalities (i.e., one or more functionalities beyond the fluorescent dye).

### SUMMARY OF THE INVENTION

According to some embodiments of the present invention, a fluorescent porous silica particle for drug delivery (i.e., a drug delivery vehicle) includes a bridged silane fluorescent dye incorporated throughout the particle's matrix. Copolymerization of a bridged silane fluorescent dye (e.g., (R′O)₃Si—R—Si(OR′)₃, where R is a fluorescent organic bridging group, and where R′ is a methyl or ethyl group) and a tetralkoxysilane (e.g., Si(OR′)₄, where R′ is a methyl or ethyl group) in the presence of a surfactant generates matrix-incorporated fluorescent porous silica particles of a predetermined size and shape. A capping layer is then bonded onto the surface of each particle and, subsequently, the surfactant within the pores of each particle is removed. The capping layer reversibly changes between closed and opened states responsive to a stimulus. A payload is then loaded within of the pores by applying the stimulus to open the capping layer. The payload is then entrapped within the pores by removing the stimulus to close the capping layer.

### BRIEF DESCRIPTION OF THE DRAWINGS

The preferred exemplary embodiments of the present invention will hereinafter be described in conjunction with the appended drawings, where like designations denote like elements.

FIG. 1 is a flow diagram of a method for producing a matrix-incorporated fluorescent porous silica particle for drug delivery in accordance with some embodiments of the present invention.

FIG. 2 is a flow diagram of a method for releasing a payload from a matrix-incorporated fluorescent porous silica particle for drug delivery in accordance with some embodiments of the present invention.

FIGS. 3A-3F depict respective stages in a prophetic example of a method for producing matrix-incorporated fluorescent porous silica particles for drug delivery in accordance with some embodiments of the present invention.

FIG. 4 depicts a method for releasing a payload from a matrix-incorporated fluorescent porous silica particle for drug delivery in accordance with some embodiments of the present invention.

### DETAILED DESCRIPTION OF THE PREFERRED EMBODIMENTS

### 1. Overview

In accordance with some embodiments of the present invention, a fluorescent porous silica particle for drug delivery (i.e., a drug delivery vehicle) includes a bridged silane fluorescent dye incorporated throughout the particle's matrix. Copolymerization of a bridged silane fluorescent dye (e.g., (R′O)₃Si—R—Si(OR′)₃, where R is a fluorescent organic bridging group, and where R′ is a methyl or ethyl group) and a tetralkoxysilane (e.g., Si(OR′)₄, where R′ is a methyl or ethyl group) in the presence of a surfactant generates matrix-incorporated fluorescent porous silica particles of a predetermined size and shape. A capping layer is then bonded onto the surface of each particle and, subsequently, the surfactant within the pores of each particle is removed. The capping layer reversibly changes between closed and opened states responsive to a stimulus. A payload is then loaded within of the pores by applying the stimulus to open the capping layer. The payload is then entrapped within the pores by removing the stimulus to close the capping layer.

### 2. Detailed Description

An organic fluorescent dye is incorporated throughout the matrix of a porous silica particle in accordance with some embodiments of the present invention. Incorporation of an organic fluorescent dye throughout a particle's matrix is highly advantageous over prior art approaches, such as incorporating an organic fluorescent dye onto the surface of a particle via surface modification or entrapping an organic fluorescent dye within a particle via core-shell encapsulation. For example, a matrix-incorporated fluorescent porous silica particle in accordance with some embodiments of the present invention can be surface modified to have aqueous dispersive properties. Moreover, a matrix-incorporated fluorescent porous silica particle in accordance with some embodiments of the present invention has zero dye leach out. In addition, the pores of a matrix-incorporated fluorescent porous silica particle in accordance with some embodiments of the present invention are available to provide further functionality, such as to carry a pharmaceutical drug molecule or other payload.

As used herein, including the claims, the term “payload” includes molecules of various sizes, shapes and functionalities including pharmaceutical drug molecules, neurotransmitters, biocides, genes, and proteins.

A matrix-incorporated fluorescent porous silica particle in accordance with some embodiments of the present invention may be, for example, a MCM-41 type (i.e., Mobil Composition of Matter No. 41) or MCM-48 type (i.e., Mobil Composition of Matter No. 48) mesoporous silica nanoparticle (MSN). The acronym “MCM” is also referred to in the art as “Mobil Crystalline Material”. MCM-type mesoporous silica materials typically possess several desirable features (e.g., stable mesoporous structures, large surface areas, tunable pore diameters and volumes, and well-defined surface properties) that make these materials ideal for hosting molecules of various sizes, shapes, and functionalities. Another advantage of MSNs is that they may be organically functionalized. Hence, in accordance with some embodiments of the present invention, the matrix-incorporated fluorescent porous silica particle may be a MSN. Matrix-incorporated fluorescent porous silica particles, including MSNs, can be generated in accordance with some embodiments of the present invention with homogenous size distribution, various shapes, and a wide spectrum of fluorescent emissions.

One skilled in the art will appreciate, however, that a matrix-incorporated fluorescent porous silica particle for drug delivery in accordance with the present invention is not limited to MSNs or MCM-type mesoporous silica materials.

Matrix-incorporated fluorescent porous silica particles for drug delivery in accordance with some embodiments of the present invention can be produced in various shapes (e.g., spheres, rods, discoids, gyroids, etc.) and in a wide range of sizes from nano to micro diameters. Such particles can be produced, for example, in homogeneous sizes.

In accordance with some embodiments of the present invention, an organic bridged fluorescent silane dye is incorporated into the silica matrix of the particle to prevent leach out. Bridged silane fluorescent dyes are bridged silane monomers with the general chemical structure of (R′O)₃Si—R—Si(OR′)₃, where the R bridging group is a fluorescent organic group and where R′ is a methyl or ethyl group. Copolymerization of the bridged silane fluorescent dye and a tetralkoxysilane (e.g., Si(OR′)₄, where R′ is a methyl or ethyl group) in the presence of a surfactant generates the matrix-incorporated fluorescent porous silica particles. Bridged silane fluorescent dyes are distributed by reaction kinetics throughout the silica matrix and, hence, bridged silane fluorescent dyes become matrix-incorporated via the copolymerization. Ordinary (i.e., non-bridged) fluorescent silane dyes, on the other hand, only surface modify the porous silica particle due to reaction kinetics. Zero dye leach out is achieved because the bridged silane fluorescent dye is matrix-incorporated into the silica matrix of the particle, and each bridged fluorescent dye molecule is matrix-incorporated with up to six points of attachment.

Organic bridged fluorescent silane dyes can be tailored to produce different emissions (typically, in either the visual spectrum or the near-infrared (NIR) spectrum) depending on which fluorescent organic group is selected as the bridging group. For example, in accordance with some embodiments of the present invention the organic bridged fluorescent silane dye is tailored to produce emissions suitable for a particular method of viewing. This tailoring is accomplished by selecting, as the bridging group of the organic bridged fluorescent silane dye, a fluorescent organic group that fluorescences within a range of wavelengths suitable for a particular method of viewing. Fluorescence can be visualized using fluorescence microscopy, UV black light (visual spectrum), night vision goggles (NIR), or other methods chosen by those skilled in the art.

The use of a matrix-incorporated fluorescent porous silica particle for use as a drug delivery vehicle, where a fluorescent dye is bound within the matrix of the silica particle and where the silica particle is surface modified in accordance with some embodiments of the present invention, is highly advantageous over prior art approaches, such as core-shell encapsulation or particle surface modification. These prior art approaches are limited by fluorescent dye leach out and cannot accommodate additional surface modification because the surface functionalities of the particle are already taken up by a dye molecule. In contrast to these prior art approaches, there is no dye leach out from a matrix-incorporated fluorescent porous silica particle in accordance with some embodiments of the present invention because the fluorescent dye is bound within the matrix of the silica particle. Also in contrast to these prior art approaches, because the surface functionalities are not already taken up by a dye molecule, the surface of a matrix-incorporated fluorescent porous silica particle in accordance with some embodiments of the present invention is available for additional surface modification to allow the particles to be dispersed in different systems.

Encapsulating a pharmaceutical drug or other payload inside the porous framework of the matrix-incorporated fluorescent porous silica particle using a capping layer (that is stimulus-responsive to open and close the pores) to physically block the drug or other payload from leaching out in accordance with some embodiments of the present invention is highly advantageous over prior art approaches, such as core-shell encapsulation or particle surface modification, that are limited by payload leach out. In contrast to these prior art approaches, there is no payload leach out from a matrix-incorporated fluorescent porous silica particle in accordance with some embodiments of the present invention because the capping layer (when stimulus is not applied) closes the pores to physically block the payload from leaching out from within the porous framework of the silica particle.

In accordance with some embodiments of the present invention, copolymerization of a fluorescent bridged bis/tris alkoxysilane with a tetralkoxysilane generates fluorescent porous silica particles of a predetermined shape and size. The copolymerization incorporates a fluorescent dye directly into the matrix of the silica particle thereby preventing dye leach out. The copolymerization reaction also leaves the surface of each particle available for subsequent modification to disperse into different media. Hence, the particles may be tailored, through surface modification, to disperse into a particular media. Because the particles are porous a drug can be added within the pores for subsequent release. The resulting particles can then be administered to a specimen and analyzed through the use of a fluorescence microscope or other fluorescence inducing instruments.

FIG. 1 is a flow diagram of a method 100 for producing a matrix-incorporated fluorescent porous silica particle for drug delivery in accordance with some embodiments of the present invention. The method 100 sets forth the preferred order of the steps. It must be understood, however, that the various steps may occur at any time relative to one another.

The method 100 begins by incorporating a bridged silane fluorescent dye throughout a matrix of a porous silica particle (step 102). In accordance with some embodiments of the present invention, step 102 is performed via a copolymerization reaction between a bridged silane fluorescent dye and a tetralkoxysilane in the presence of a surfactant. For example, a matrix-incorporated fluorescent porous silica particle in accordance with some embodiments of the present invention may be manufactured using a modified Lai et al. synthesis to incorporate a bridged silane fluorescent dye into the matrix of the silica material. The Lai et al. synthesis, itself, is well known in the art. The Lai et al. synthesis is, for example, disclosed in Lai et al., “A Mesoporous Silica Nanosphere-Based Carrier System with Chemically Removable CdS Nanoparticle Caps for Stimuli-Responsive Controlled Release of Neurotransmitters and Drug Molecules”, J. Am. Chem. Soc. 2003, Vol. 125, Pages 4451-4459, which is hereby incorporated herein by reference in its entirety.

In accordance with some embodiments of the present invention, the Lai et al. synthesis is modified to introduce a bridged silane fluorescent dye into the copolymerization reaction to incorporate the bridged silane fluorescent dye throughout the matrix of the resultant porous silica particles. In accordance with some embodiments of the present invention, the bridged silane fluorescent dye has the general chemical structure (R′O)₃Si—R—Si(OR′)₃, wherein R is a fluorescent organic bridging group, and wherein R′ is a methyl or ethyl group. Bridged silane fluorescent dyes may be readily synthesized using processes well known in the art. For example, the synthesis of a myriad of bridged silane fluorescent dyes is disclosed in U.S. Pat. No. 7,777,176 B2, issued to Loy et al. on Aug. 17, 2010, entitled “COMPOSITION AND METHOD TO CHARACTERIZE MEMBRANES' DEFECTS”, which is hereby incorporated herein by reference in its entirety.

The bridged silane fluorescent dye can be added throughout the silica matrix through copolymerization of a tetralkoxysilane and a bridged silane fluorescent dye in the presence of a surfactant. The tetralkoxysilane in step 102 may be, for example, tetraethyl orthosilicate (TEOS) or any other suitable tetralkoxysilane (e.g., Si(OR′)₄, where R′ is a methyl or ethyl group).

The surfactant in step 102 may be, for example, cetyltrimethylammonium bromide (CTAB) or any other suitable surfactant. The surfactant is referred to as a “template surfactant” because it serves as a template for the pores of the porous matrix-incorporated fluorescent silica nanoparticles. That is, the pore diameter can be controlled by employing surfactants with different chain lengths. In accordance with some embodiments of the present invention, the matrix-incorporated fluorescent porous silica particle is a nanoparticle or microparticle approximately 100 to 2000 nm in diameter, and the pores of the silica particle have an average diameter of approximately 1.5 to 20 nm.

One example of a bridged silane fluorescent dye that could be used in step 102 is 4,4′-bis(4-(triethoxysilyl)styryl)biphenyl, which can easily be incorporated into the Lai et al. synthesis. Other bridged silane fluorescent dyes both in the visual spectrum and near-infrared (NIR) spectrum may be also chosen by those skilled in the art. Suitable fluorescent dyes include, but are not limited to, monomers with the general chemical structure of (R′O)₃Si—R—Si(OR′)₃, where the R bridging group is a fluorescent organic group and where R′ is a methyl or ethyl group. The amount of bridged silane fluorescent dye can be varied but generally less than 1.0 mol % is acceptable. Particle size can be controlled by stir speed.

The method 100 continues by bonding a capping layer onto a surface of the porous silica particle (step 104). In step 104, the surface of each porous silica particle synthesized in step 102 is modified with a polymer for capping the pores. In accordance with some embodiments of the present invention, the capping layer reversibly changes from a first state (pores closed) to a second state (pores open) responsive to application of a stimulus, such as temperature, light, pH, and the like.

After synthesizing the particles in step 102, in accordance with some embodiments of the present invention, with the surfactant (e.g., CTAB) still in the pores, step 104 is performed in two stages. In the first stage of step 104, the surface of the particles can be modified with an allyl- or vinylalkoxy- or a chloro-silane functionality. For example, the surface of the particles may be vinyl-functionalized by placing the particles in a solution containing a suitable vinylalkoxysilane (e.g., vinyltriethoxysilane, vinyltrimethoxysilane, and the like) and suitable solvent (e.g., ethanol, methanol, and the like). Alternatively, the unsaturated functionality could be copolymerized during the sol gel polymerization. In the second stage of step 104, utilizing a polymerize through approach, the surface of the particle can be modified with a polymer capping layer that allows for pore closing/opening. An example of a suitable polymer for use as the polymer capping layer is poly(N-isopropylacrylamide) or copolymers thereof. The polymer capping layer can be polymerized using conventional radical polymerization or Reversible Addition-Fragmentation chain Transfer (RAFT) can be utilized to polymerize the polymer capping layer onto the surface of the particle. Poly(N-isopropylacrylamide) (PNIPA) is a temperature responsive polymer that when heated above 32° C. undergoes a change from a swollen state to a dehydrated state (˜90% of its mass is lost), thus allowing for the release of the pharmaceutical drug. Other surface modification functionalities, polymers and/or capping methods may be chosen by those skilled in the art.

The method 100 continues by removing the surfactant within pores of the porous silica particle (step 106). In step 106 the surfactant is removed to allow the pores to be filled with a pharmaceutical drug or other payload. For example, after the surface of the particle has been modified with the polymer capping layer (e.g., PNIPA) in step 104, the surfactant (e.g., CTAB) is removed in step 106 to open the pores for accepting the pharmaceutical drug or other payload.

The method 100 continues by loading a payload within of the pores of the porous silica particle (step 108). In accordance with some embodiments of the present invention, step 108 is performed by applying the stimulus to place the capping layer in the second state (pores open). As an example, vancomycin, a drug used to treat colitis, can be integrated into the pores in step 108 by heating the PNIPA-coated particles above 32° C. Other pharmaceutical drugs or payloads may be chosen by those skilled in the art.

The method 100 continues by entrapping the payload within the pores of the porous silica particle (step 110). In accordance with some embodiments of the present invention, step 110 is performed by removing the stimulus to place the capping layer in the first state (pores closed). For example, after the drug (e.g., vancomycin) or other payload is integrated into the pores in step 108, the drug or other payload can be entrapped within the pores in step 110 by quenching the PNIPA-coated particles at less than 32° C.

Additionally, the polymer capping layer (e.g., PNIPA) can be modified to allow the particles to be disbursed in different media. If such additional surface modification is desired, the method 100 continues by bonding a surface modifier onto a surface of the capping layer (step 112). For example, the surface of the particles can be additionally modified using a poly(ethylene glycol) (PEG) silane to allow the particles to be disbursed in an aqueous media. Other surface modifiers may be chosen by those skilled in the art.

Surface modification of the particles with PEG may make the drug delivery vehicle non-toxic for in vivo.

Surface modification of the particles with PEG may also serve to increase the Enhanced Permeability and Retention (EPR) effect. Typically, PEG surface modification of silica nanoparticles increases the EPR effect. The EPR effect is the property by which certain sizes of molecules (typically liposomes, nanoparticles, and macromolecular drugs) tend to accumulate in tumor tissue much more than those same molecules tend to accumulate in normal tissue. Matrix-incorporated fluorescent porous silica particles for drug delivery in accordance with some embodiments of the present invention may be tailored in size to be site-selective. Moreover, this site-selectivity may be enhanced via surface modification of the particles with PEG.

FIG. 2 is a flow diagram of a method 200 for releasing a payload from a matrix-incorporated fluorescent porous silica particle for drug delivery in accordance with some embodiments of the present invention. The method 200 sets forth the preferred order of the steps. It must be understood, however, that the various steps may occur at any time relative to one another.

The method 200 begins by providing a matrix-incorporated fluorescent porous silica particle for drug delivery (step 202). For example, such a drug delivery vehicle may be produced using method 100 (shown in FIG. 1).

The method 200 continues by applying a stimulus to release the payload from the pores of the matrix-incorporated fluorescent porous silica particle for drug delivery (step 204). For example, a drug (e.g., vancomycin) may be released from the pores of PNIPA-coated particles in step 204 by heating the particles above 32° C. (e.g., the particles may be injected or otherwise administered into a human patient, whereupon the drug is released via the body heat of the human patient). One skilled in the art will appreciate that other methods of drug release (e.g., through the application of one or more stimuli, such as temperature, light, pH, and the like) are possible depending on how the drug is capped in the pores.

The method 200 continues by visualizing the fluorescence of the matrix-incorporated fluorescent porous silica particle for drug delivery (step 206). Fluorescence can be visualized using fluorescence microscopy, UV black light (visual spectrum), night vision goggles (NIR), or other methods chosen by those skilled in the art.

FIGS. 3A-3F depict respective stages in a prophetic example of a method for producing matrix-incorporated fluorescent porous silica nanoparticles for drug delivery in accordance with some embodiments of the present invention.

### Preparation of Matrix-Incorporated Fluorescent Porous Silica Nanoparticles

FIG. 3A depicts a matrix-incorporated fluorescent porous silica nanoparticle preparation stage in a prophetic example of a method for producing matrix-incorporated fluorescent porous silica nanoparticles for drug delivery in accordance with some embodiments of the present invention. Particles are prepared through a modified Lai et al. synthesis using n-cetyltrimethylammonium bromide (CTAB), sodium hydroxide (NaOH), deionized water, tetraethyl orthosilicate (TEOS), and a bridged silane fluorescent dye (i.e., in this prophetic example, 4,4′-bis(4-(triethoxysilyl)styryl)biphenyl). CTAB and TEOS are commercially available from chemical suppliers, such as Sigma-Aldrich, St. Louis, Mo. An exemplary synthesis of the bridged silane fluorescent dye used in this prophetic example is described below.

CTAB (1.00 g, 2.74×10⁻³ mol) is dissolved in deionized water (480 mL) in a 1000 mL round bottom flask with a condenser. In this prophetic example, CTAB is a template surfactant. Aqueous NaOH (2.00M, 3.50 mL) is then added to the CTAB solution, followed by raising the temperature of this surfactant solution to 80° C. using an oil bath.

TEOS (6.2 mL, 2.78×10⁻² mol) and the bridged silane fluorescent dye (0.01-1.0 mol %) are then added to the surfactant solution. In this prophetic example, 4,4′-bis(4-(triethoxysilyl)styryl)biphenyl (175.5 mg, 2.57×10⁻⁴ mol) is used as the bridged silane fluorescent dye. The mixture is allowed to stir for 2 h to give rise to white precipitate. The solid white product is filtered, washed with deionized water and methanol, and dried in air. The bridged silane fluorescent dye used in this prophetic example (i.e., 4,4′-bis(4-(triethoxysilyl)styryl)biphenyl) may be synthesized as discussed below.

### Synthesis of Bridged Silane Fluorescent Dye

The bridged silane fluorescent dye used in this prophetic example (i.e., 4,4′-bis(4-(triethoxysilyl)styryl)biphenyl) is synthesized using (triethoxysilyl)styrene, 4,4′-dibromephenyl, anhydrous toluene, tri-o-tolylphosphine, and palladium acetate. (Triethoxysilyl)styrene, 4,4′-dibromephenyl, tri-o-tolylphosphine, and palladium acetate are commercially available from chemical suppliers, such as Sigma-Aldrich, St. Louis, Mo. To a three neck 25 mL round bottom flask, (triethoxysilyl)styrene (1.006 g, 3.8 mmol) is added. To this solution, a magnetic stir bar and 4,4′-dibromephenyl (1.176 g, 3.8 mmol) are added, followed by anhydrous toluene (10 mL). To this solution, tri-o-tolylphosphine (0.085 g, 2.6×10⁻⁵ mol) along with palladium acetate (0.015 g, 4.45×10⁻⁵ mol) are added. This solution is stirred and heated at 105° C. After an hour, an additional amount of (triethoxysilyl)styrene (0.9006 g, 3.4 mmol) is added along with additional anhydrous toluene (10 mL). The solution is allowed to react for 24 h. The brown solution is vacuum suctioned through CELITE® to remove palladium. The solution is removed in vacuo yielding 4,4′-bis(4-(triethoxysilyl)styryl)biphenyl, a yellow solid.

### Vinyl-Functionalized Surface Modification of Matrix-Incorporated Fluorescent Porous Silica Nanoparticles

FIG. 3B depicts a vinyl-functionalized surface modification stage in a prophetic example of a method for producing matrix-incorporated fluorescent porous silica nanoparticles for drug delivery in accordance with some embodiments of the present invention. As-synthesized dried particles (100 mg) with surfactant still in the pores are placed in ethanol (10 mL) with vinyltriethoxysilane (3 mg, 1.58×10⁻⁵ mol) and refluxed for 24 h. Vinylethoxysilane is commercially available from chemical suppliers, such as Sigma-Aldrich, St. Louis, Mo. Vinyl-modified porous silica particles are then washed with ethanol and methanol and finally dried at 80° C. for 3 h in vacuo.

### PNIPA Surface Modification of Matrix-Incorporated Fluorescent Porous Silica Nanoparticles

FIG. 3C depicts a PNIPA surface modification stage in a prophetic example of a method for producing matrix-incorporated fluorescent porous silica nanoparticles for drug delivery in accordance with some embodiments of the present invention. Dried vinyl-modified porous silica particles (70 mg) are placed in a round bottom flask containing dimethylformamide (DMF) (1.5 mL), azobisisobutyronitrile (AIBN) (6.57×10⁻⁵ g, 0.0004 mmol), 2-cyanoprop-2-yl-dithiobezoate (CPDB) (1.20×10⁻³, 0.0054 mmol) and reacted for 48 h at 60° C. DMF, AIBN and CPDB are commercially available from chemical suppliers, such as Sigma-Aldrich, St. Louis, Mo. After reaction, solvent is removed and particles are dried in vacuo.

### Removal of Surfactant from Matrix-Incorporated Fluorescent Porous Silica Nanoparticles

FIG. 3D depicts a surfactant removal stage in a prophetic example of a method for producing matrix-incorporated fluorescent porous silica nanoparticles for drug delivery in accordance with some embodiments of the present invention. To remove the surfactant template (CTAB), as-synthesized particles (100 mg) are refluxed for 24 h in a solution of HCL (9.00 mL, 37.4%) and methanol (160 mL), followed by extensive washes with deionized water and methanol. The resulting surfactant-removed silica nanoparticles are dried in vacuo to remove remaining solvent in the mesopores.

### Loading Pharmaceutical Drug within Pores of Matrix-Incorporated Porous Fluorescent Silica Nanoparticles

FIG. 3E depicts a pharmaceutical drug loading stage in a prophetic example of a method for producing matrix-incorporated fluorescent porous silica nanoparticles for drug delivery in accordance with some embodiments of the present invention. Purified matrix-incorporated fluorescent porous silica nanoparticles (100 mg) with PNIPA surface modification are incubated in a PBS buffer solution (0.60 mL, pH 7.4) of vancomycin (3.00 μmol) for 24 h at 40° C. Vancomycin hydrochloride (also referred to herein as “vancomycin”) is commercially available from chemical suppliers, such as Sigma-Aldrich, St. Louis, Mo. The PBS (phosphate buffered saline) buffer solution (10.00 mM, pH 7.4) may be prepared earlier with the total ionic strength of 0.06 M.

### Entrapping Pharmaceutical Drug within Pores of Matrix-Incorporated Porous Fluorescent Silica Nanoparticles

FIG. 3F depicts a pharmaceutical drug entrapping stage in a prophetic example of a method for producing matrix-incorporated fluorescent porous silica nanoparticles for drug delivery in accordance with some embodiments of the present invention. After incubating for 24 h at 40° C., the solution is quenched to below 32° C. to entrap the vancomycin within the pores of the particles. Resulting particles are isolated and dried below 32° C.

### PEG Surface Modified Porous Matrix-Incorporated Fluorescent Silica Nanoparticles

In order to make the drug delivery vehicle non-toxic for in vivo, the surface can be additionally modified with polyethylene glycol (PEG). Dried particles (100 mg) are dispersed in a solution of methoxy-PEG-silane (3 mM) in toluene (5 mL) containing 2M Ammonia (catalyst) added dropwise until the pH 9. Particles are allowed to stir for 4 h at 60° C. Methoxy-PEG-silane is commercially available from chemical suppliers, such as Sigma-Aldrich, St. Louis, Mo. The solution is then filtered and extensively washed with toluene and ethanol, and dried in vacuo.

FIG. 4 depicts a method for releasing a payload from a matrix-incorporated fluorescent porous silica particle for drug delivery in accordance with some embodiments of the present invention. The payload (e.g., vancomycin) is released from the pores of PNIPA-coated particle by heating the particle above 32° C. For example, the particle may be injected or otherwise administered into a human patient, whereupon the payload is released via the body heat of the human patient. Fluorescence can be visualized using fluorescence microscopy, UV black light (visual spectrum), night vision goggles (NIR), or other methods chosen by those skilled in the art.

One skilled in the art will appreciate that many variations are possible within the scope of the present invention. Thus, while the present invention has been particularly shown and described with reference to particular embodiments thereof, it will be understood by those skilled in the art that these and other changes in form and detail may be made therein without departing from the spirit and scope of the present invention.

What is claimed is:

1. A porous silica particle for drug delivery, the porous silica particle comprising:
a bridged silane fluorescent dye incorporated throughout a matrix of the porous silica particle via a copolymerization reaction between a bridged silane fluorescent dye and a tetralkoxysilane in the presence of a surfactant, wherein the bridged silane fluorescent dye has the general chemical structure (R′O)₃Si—R—Si(OR′)₃, wherein R is a fluorescent organic bridging group, and wherein R′ is a methyl or ethyl group;
a payload contained within pores of the porous silica particle;
a capping layer bonded onto a surface of the porous silica particle, wherein the capping layer is responsive to a stimulus to release the payload from the pores of the porous silica particle.

2. The porous silica particle as recited in claim 1, wherein the bridged silane fluorescent dye comprises 4,4′-bis(4-(triethoxysilyl)styryl)biphenyl and the tetralkoxysilane comprises tetraethyl orthosilicate (TEOS).

3. The porous silica particle as recited in claim 1, wherein the porous silica particle is a nanoparticle or microparticle approximately 100 to 2000 nm in diameter, and wherein the pores of the silica particle have an average diameter of approximately 1.5 to 20 nm.

4. The porous silica particle as recited in claim 1, wherein the capping layer comprises poly(N-isopropylacrylamide).

5. The porous silica particle as recited in claim 1, wherein the capping layer is responsive to at least one of a temperature stimulus, a light stimulus, and a pH stimulus to release the payload from the pores of the silica particle.

6. The porous silica particle as recited in claim 1, further comprising:
a surface modifier bonded onto a surface of the capping layer.

7. The porous silica particle as recited in claim 6, wherein the surface modifier is selected to increase the dispersion of the porous silica particle in an aqueous media.

8. The porous silica particle as recited in claim 7, wherein the surface modifier comprises poly(ethylene glycol).

9. A method of synthesizing a porous silica particle for drug delivery, comprising the steps of:
incorporating a bridged silane fluorescent dye throughout a matrix of the porous silica particle via a copolymerization reaction between a bridged silane fluorescent dye and a tetralkoxysilane in the presence of a surfactant, wherein the bridged silane fluorescent dye has the general chemical structure (R′O)₃Si—R—Si(OR′)₃, wherein R is a fluorescent organic bridging group, and wherein R′ is a methyl or ethyl group;
bonding a capping layer onto a surface of the porous silica particle, wherein the capping layer reversibly changes from a first state to a second state responsive to a stimulus;
removing the surfactant within pores of the porous silica particle;
loading a payload within of the pores of the porous silica particle, wherein the step of loading a payload within the pores of the silica particle comprises the step of applying or removing the stimulus to place the capping layer in the first state;
entrapping the payload within the pores of the porous silica particle, wherein the step of entrapping the payload within the pores of the porous silica particle comprises the step of removing or applying the stimulus to place the capping layer in the second state.

10. The method as recited in claim 9, wherein the bridged silane fluorescent dye comprises 4,4′-bis(4-(triethoxysilyl)styryl)biphenyl and the tetralkoxysilane comprises tetraethyl orthosilicate (TEOS).

11. The method as recited in claim 9, wherein the capping layer comprises poly(N-isopropylacrylamide).

12. The method as recited in claim 11, wherein the step of bonding a capping layer onto a surface of the porous silica particle comprises the steps of:
vinyl-functionalizing the surface of the porous silica particles; and
placing the porous silica particles having a vinyl-functionalized surface into a solution containing dimethylformamide (DMF), azobisisobutyronitrile (AIBN) and 2-cyanoprop-2-yl-dithiobenzoate (CPDB).

13. The method as recited in claim 9, wherein the capping layer reversibly changes from the first state to the second state responsive to at least one of a temperature stimulus, a light stimulus, and a pH stimulus.

14. The method as recited in claim 9, wherein the step of loading a payload within of the pores of the porous silica particle comprises incubating the porous silica particles with the surfactant removed in a phosphate buffered saline (PBS) solution containing a drug and heating the PBS solution to a temperature at or above a predetermined temperature sufficient to place the capping layer in the first state.

15. The method as recited in claim 14, wherein the step of entrapping the payload within the pores of the porous silica particle comprises quenching the porous silica particles with the drug within the pores to a temperature at or below a predetermined temperature sufficient to place the capping layer in the second state.

16. The method as recited in claim 9, further comprising the step of:
bonding a surface modifier onto a surface of the capping layer.

17. The method as recited in claim 16, wherein the step of bonding a surface modifier onto a surface of the capping layer comprises dispersing the porous silica particle in a solvent and adding a silane-based surface modifier.

18. The method as recited in claim 17, wherein the solvent comprises toluene and the silane-based surface modifier comprises methoxy-poly(ethylene glycol)-silane.

19. A method of releasing a payload from a porous silica particle for drug delivery, comprising the steps of:
providing a porous silica particle, comprising:
a bridged silane fluorescent dye incorporated throughout a matrix of the porous silica particle via a copolymerization reaction between a bridged silane fluorescent dye and a tetralkoxysilane in the presence of a surfactant, wherein the bridged silane fluorescent dye has the general chemical structure (R′O)₃Si—R—Si(OR′)₃, wherein R is a fluorescent organic bridging group, and wherein R′ is a methyl or ethyl group;
a payload contained within pores of the porous silica particle;
a capping layer bonded onto a surface of the porous silica particle, wherein the capping layer is responsive to a stimulus to release the payload from the pores of the porous silica particle;
applying the stimulus to release the payload from the pores of the porous silica particle.

20. The method as recited in claim 19, wherein the step of applying the stimulus to release the payload from the pores of the porous silica particle comprises changing the capping layer from a closed state to an open state by applying at least one of a temperature stimulus, a light stimulus, and a pH stimulus to the porous silica particle.
